(19) United States
(12) Patent Application Publication
(10) Pub. No.: US 2010/0029581 A1
(43) Pub. Date: Feb. 4, 2010

(54) NUTRITIONAL SUPPLEMENT

(76) Inventor: Ramandeep Kaur Dhillon-Gill, Wooster, OH (US)

Correspondence Address: HAHN LOESER & PARKS, LLP, One GOJO Plaza, Suite 300, AKRON, OH, 44311-1076, US

(21) Appl. No.: 12/183,701

(22) Filed: Jul. 31, 2008

(51) Int. Cl. A61K 31/20; A61K 31/7016; A61K 31/7004; A61P 3/02; A61K 31/70

(52) U.S. Cl. 514/53; 514/560; 514/23

(57) ABSTRACT

A nutritional supplement comprises about 2 parts roasted, ground flaxseed, about 2 parts of chick pea flour, about 1.5-2 parts of whole wheat or brown rice flour, about 1 part of raw, ground almonds and optionally, about 0.5-1 part of whole wheat bran. The supplement may additionally contain one or both of raw blue agave nectar and sunflower oil. The supplement may also contain cardamom and/or ginger. The nutritional supplement comprises at least about 1.5 grams of Omega-3 fatty acids, less than about 1.5 grams (g) of glucose, and less than about 0.6 g of sucrose in a serving of about 2 ounces (about 59 ml). A method of supplementing the nutrition of an individual comprises administering the nutritional supplement to the individual.

### BACKGROUND OF THE INVENTION

This invention relates to nutritional supplements. In the field of nutrition, various beneficial effects are attributed to different foods and supplements. For example, it is widely recognized that “healthy” oils such as fish oil and flaxseed oil, are sources of relatively high concentrations of omega-3, omega-6, and omega-9 fatty acids. These fatty acids are widely recognized as providing beneficial effects on total cholesterol and triglycerides and thereby improving cardiovascular health. The American Cardiology Association recommends the consumption of anywhere from approximately 1 gram to 4 gram of Omega-3 fatty acid per day, depending on a patient's underlying lipid profile. Omega-3 and omega-6 fats are also believed to have an anti-inflammatory effects and it has been observed that at least some people with coronary heart disease have underlying inflammation. Evidence of this is provided by an elevated blood protein called C-reactive protein. Omega fatty acids are also known to have other health benefits including relief from pain syndromes through a process called thermogenics which, in turn, may help with suppressing appetitie.

Omega-3 fatty acids cannot be made or converted from any other source of fat in the human body. It must be consumed. While fish oil is a good source of omega-3 fatty acids, the best sources of omega-3 fatty acids are typically oily fish such as salmon, herring, cod, mackerel or sardines. Some individuals do not find these types of fish appealing and tend not to consume them as much as necessary to get the required amount of omega-3 fats. Other people will simply not eat any type of fish. Other types of fish rich in omega-3 fatty acids, such as swordfish, albacore tuna, and marlin, have a higher chance of containing harmful levels of dioxins and mercury, and therefore, their intake should be more limited. Furthermore, those who take fish oil supplements often take them in the form of an enteric coated tablet because of their unpleasant taste and aftertaste.

Flaxseed also has thermogenic and anti-inflammatory properties. A thermogenic effect, as it pertains to food, is a term used to describe the way that certain foods can increase the amount of heat generated as the food is digested, resulting in an increased metabolism for a period of time. At its most basic level, the thermogenic effect is a result of the body having to expend energy to digest food. Some sources of energy from food are less available than others, and thereby require more energy to make it of use in the body. Protein is one particular type of food with a high thermogenic effect on a macronutrient level. It has been estimated that 27 percent of the calories (on average) that a human being consumes from protein are used in the process of digesting the protein. Most of this is attributed to the fact that the protein has to be broken down into amino acids which can then be made use of by the body. This is a labor-intensive process intracellularly. For example, if an individual consumes 300 calories in pure protein (such as a purified protein powder, egg whites or the like), the thermogenic effect would consume 81 calories during the act of digestion, resulting in a net gain of only 219 calories. This has been the basis of popular high protein diets such as the Adkins diet.

Flaxseed is considered beneficial for relief from certain types of pain because of its anti-inflammatory properties. Fibromyalgia and osteoarthritis are not considered inflammatory-type of pain syndromes (unlike rheumatoid arthritis) in their initial stages, but over time inflammation develops, as with many medical conditions. People with fibromyalgia wake up in the morning with pain and often describe the discomfort as the inability to move about as the “dry sponge from the kitchen sink.” This is also why people with fibromyalgia are advised to drink ample amounts of water. Anything that dehydrates them, such as caffeine or alcohol, is not recommended for these patients. The short effect of morning coffee indeed has thermogenic and anti-inflammatory properties, however, it dehydrates them even further because of the diuretic effect of caffeine. Alcohol also seems to give the patient an illusion that it creates warmth and lubrication type of feeling in joints and muscles.

Additionally, the National Cholesterol Education Program (NCEP) recommends a dietary fiber intake of about 20-25 grams/day. This is suggested as a lifestyle modification for all patients.

Therefore, there is a need for a nutritional supplement that provides health benefits such as those provided by the omega fats. It would be an added benefit if this supplement came in a food like preparation that was pleasant tasting, pure and preservative-free. There is also a need for such a supplement that also provides fiber but is also free of a significant amount of gluten.

### SUMMARY OF THE INVENTION

It is an aspect of the present invention to provide a nutritional supplement providing a thermogenic effect and health benefits from omega-3 and omega-6 fatty acids.

In general, the present invention provides a nutritional supplement comprising about 2 parts roasted, ground flaxseed, about 2 parts of chick pea (garbanzo bean) flour, about 1.5-2 parts of flour selected from whole wheat flour or brown rice flour, about 1 part of raw, ground almonds and optionally, about 0.5-1 part of whole wheat bran. The supplement may additionally contain one part blue agave nectar as a sweetener. It also may contain 1.5-1.8 parts of sunflower oil. The liquid “parts” have the same proportion to the “dry” parts as the proportion of liquid cups to dry cups. The supplement may also contain ground cardamom and ground ginger for example in the amounts of about 0.0025-0.005 parts cardamom and about 0.01 parts ginger. In one particular embodiment, the components are organically grown.

In another embodiment, the nutritional supplement consists of or at least consists essentially of about 2 parts roasted, ground flaxseed, about 2 parts of chick pea flour, about 1.5-2 parts of whole wheat flour, about 0.5-1 part of whole wheat bran, about 1 part of raw, ground almonds, about 0.0025 parts cardamom, about 0.01 parts ginger, about one part raw blue agave nectar, about 1.75 parts of sunflower oil, where the liquid “parts” have the same proportion to the dry “parts” as the proportion of liquid cups to dry cups.

In another embodiment, the nutritional supplement consists of or at least consists essentially of about 2 parts roasted, ground flaxseed, about 2 parts of chick pea flour, about 2 parts of brown rice flour, about 1 part of raw, ground almonds, about 0.0025 parts cardamom, about 0.01 parts ginger, about one part raw blue agave nectar, about 1.5 parts of sunflower oil, where the liquid “parts” have the same proportion to the dry “parts” as the proportion of liquid cups to dry cups. In that embodiment, the nutritional supplement does not contain whole wheat flour, wheat bran or any other source of gluten, to provide a gluten-free nutritional supplement.

A nutritional supplement comprises about 1.5-1.7 grams of Omega-3 fatty acids. There is roughly an average of 4.6 grams of Omega-6 in one serving (about 2 ounces (ozs) or about 59 milliliters (ml)) of the nutritional supplement. This nutritional supplement provides about 3.6 grams of dietary fiber in a 2 ounce (about 59 ml) serving. A majority of the simple sugars are from fructose (desired source of sugars) and a minority are from the other less healthy sugars, especially from a diabetic patient's perspective, i.e., glucose and sucrose. The supplement has roughly 5 grams of fructose to about 2 grams of “other simple sugars,” including less than about 1.5 grams (g) of glucose, and less than about 0.6 g of sucrose in a serving of about 2 ounces (about 59 ml). In one embodiment, the nutritional supplement provides at least about 1.7 grams of Omega-3 fatty acids in a serving of about 2 ounces (about 59 ml).

A method of supplementing the nutrition of an individual is also provided. The method comprises administering a nutritional supplement to the individual, where the nutritional supplement comprises at least about 1.5 grams of Omega-3 fatty acids and contains less than about 1.5 grams (g) of glucose, and less than about 0.6 g of sucrose in a serving of about 2 ounces (about 59 ml). The nutritional supplement may comprise one or more components selected from the group consisting of chick pea flour, whole wheat flour, brown rice flour, whole wheat bran and ground almonds. The nutritional supplement may additionally comprise at least one of raw blue agave nectar, and sunflower oil. Additionally, the supplement may additionally comprise cardamom, ginger, or both. The supplement has a pleasant taste and may therefore be unencapsulated.

### DETAILED DESCRIPTION OF THE INVENTION

The present invention is directed toward a nutritional supplement that provides a thermogenic effect and health benefits from omega-3 and omega-6 fatty acids. The following examples should not be viewed as limiting the scope of the invention. The claims will serve to define the inventions.

In one embodiment, the nutritional supplement comprises flaxseed that has been roasted and ground, garbanzo (chick pea) flour, whole wheat flour, sunflower oil, ground almonds, agave nectar, wheat bran, ginger and cardamom. In an alternate embodiment, brown rice flour is substituted for the whole wheat flour, the wheat bran is omitted and no other source of gluten is added, for a gluten-free nutritional supplement. Each of these components provides a nutritional benefit and may be an organically grown product to minimize or eliminate the negative effects of artificial pesticides, fertilizers and the like.

The roasted and ground flaxseed provides a rich source of omega-3 and omega-6 fatty acids. The flaxseeds are slightly roasted to preserve as much of the nutritional properties of the seeds as possible. Flaxseed, via the omega-3 benefits, is used for the prevention of coronary artery disease and the treatment of high cholesterol and triglycerides. Flaxseed is believed to be particularly beneficial in raising levels of HDL (“good cholesterol”) and in lowering levels of LDL (“bad cholesterol”). Raising HDL levels is considered to be more difficult than lowering LDL levels. Statin drugs for treatment of hyperlipidemia do a good job lowering the LDL cholesterol but HDL cholesterol has always remained a challenge for the patient and the treating physician. Flaxseed is also very beneficial in lowering total triglycerides. High triglycerides and low HDL are usually found in diabetic patients and has its own pathogenesis in coronary heart disease which is very different than patients who have high cholesterol with the major problem being high LDL cholesterol.

Flaxseed is also considered to have anti-inflammatory properties and is therefore useful for patients with fibromyalgia, and/or osteoarthritis, even though these conditions are not considered inflammatory-type of pain syndromes initially. The inflammation develops slowly as the “arthritis getting worse”, at least initially. Over time, however, inflammation becomes apparent. The thermogenic effect of flaxseed provides lubrication benefits to muscles and tendons, thereby increasing flexibility. The thermogenic properties of flaxseed are also believed to help people with increasing their metabolic rate and assist with weight loss, provided the portion size of the nutritional supplement is controlled.

Magnesium has been considered to be a very beneficial electrolyte in muscle relaxation process and therefore an important sleep aid. This is important for fibromyalgia patients because sleep disturbance is a common feature of fibromyalgia. In the nutritional supplement of the present invention, almonds provide a good source of magnesium. Fibromyalgia patients are frequently advised to take a magnesium/calcium/vitamin D combination supplement already. Magnesium also helps to deter insulin resistance tendency in an individual with metabolic syndrome who is predisposed to Type II diabetes. Frequently, these patients are also getting calcium-magnesium-Vitamin D combination supplements. Vitamin D is needed to absorb calcium. The present composition provides Vitamin D and magnesium.

Blue Agave Nectar is a natural product that is derived from the carbohydrate present in the agave plant through a totally natural, thermic (heat) process with no chemicals involved. The thermic process breaks down the carbohydrates into sugars, mainly fructose. Fructose is a simple sugar found mainly in fruit and vegetables. Agave nectar has glycemic index (GI) of only 11, whereas table sugar has glycemic index of 68-85 depending on the level of refining of the table sugar. Consumption of foods with a low glycemic index is desirable for patients with, or at risk of developing, diabetes. With low GI foods, a rapid increase or “spike” in a person's blood sugar level is not seen. Rather, a steady, prolonged release of sugar into the bloodstream is observed. When foods with a high GI are consumed, however, a rise in blood sugar is observed together with insulin release by the pancreas. The insulin removes sugar from the blood and excess calories from the sugar are eventually stored as fat. This process results in low blood sugar which stimulates hunger. The process of insulin-resistance develops when a person has a habit of feeding themselves high GI foods. It should be no surprise, then, that sugar consumption leads to obesity and hypoglycemia (low blood sugar), and diabetes. On the other hand, with low G.I. foods like Agave nectar, lower levels of insulin are released from the pancreas. Additionally, almonds are reputed to assist with decreasing the likelihood of developing insulin resistance.

Sunflower oil is from a non-GMO (Genetically Modified Organism) and is not a hydrogenated oil. Its neutral flavor makes it excellent for general cooking as well as for light sautéing, sauces and salads. It is considered one of the best oils to be used for cooking because of its non-genetically modified nature. Genetic modified foods are considered undesirable in the natural health food industry. In contrast, canola oil has received scrutiny recently for this reason.

Those who are vegetarians frequently need to find sources of lean proteins in their diet. In the present nutritional supplement, chick pea (garbenzo bean) flour and the whole grain of wheat provide a source of lean protein. (The endosperm portion of the wheat grain is the only source of protein in the wheat grain itself). The bran and germ portions of the wheat grain have B vitamins, phytonutrients, and trace elements as well as fiber. Additionally, the presence of complex carbohydrates and dietary fiber is generally desirable in a diet. an intake of dietary fiber of 20-25 grams/day is recommended in an average person who is starting with therapeutic lifestyle changes in their diet.

The nutritional supplement has high levels of complex carbohydrates without preservatives, cholesterol or trans fats.

In one embodiment, the nutritional supplement provides at least about 1.5 grams of Omega-3 fatty acids in a daily dose. A daily dose may be about 2 ounces (about 59 ml) or about ⅓ of a cup. In another embodiment, the nutritional supplement provides about 1.7 grams of Omega-3 fatty acids in a daily dose. In still another embodiment, the nutritional supplement provides at least about 4 grams of Omega-6 fatty acids in a daily dose. In yet another embodiment, the nutritional supplement provides about 4.9 grams of Omega-6 fatty acids in a daily dose. The supplement may contain 277 calories, 160 of which are from fat. Stated differently, the supplement may provide about 18 grams (g) of total fat, with about 1.67 g from saturated fat, about 8.9 g from monounsaturated fat and about 6.6 g from polyunsaturated fat. The supplement may also provide about 22.33 g of total carbohydrates. Of these, about 15.17 g may be complex carbohydrates, and simple sugars may account for the following amounts: about 5.15 g of fructose, about 1.45 g of glucose, and about 0.56 g of sucrose. Dietary fiber may be present in an amount of about 3.6 g per serving. In one embodiment, the supplement may also provide about 7 g of protein and the following vitamins and minerals:

| | | |
|---|---|---|
| Iron | 2.65 | milligrams (mg) |
| Magnesium | 71 | mg |
| Calcium | 40 | mg |
| Sodium | 6.5 | mg |
| Potassium | 206 | mg |
| Vitamin A | 23.8 | I.U. |
| Vitamin D | 3.0 | I.U. |
| Vitamin C | 1.0 | mg |
| Phosphorus | 138 | mg |

The nutritional supplement may be made as follows. Flaxseed is roasted and then ground. About 2 cups of the roasted and ground flaxseed is mixed with about 2 cups of chick pea (garbanzo bean) flour, about 1.5-2 cups of whole wheat flour, about 0.5-1 cup of whole wheat bran, about 1 cup of raw, ground almonds, about 1 cup of raw blue agave nectar, and about 1.75 cups of sunflower oil. About 0.125 teaspoons of cardamom and about 0.5 teaspoons of ginger may also be added.

In another embodiment, brown rice flour replaces the whole wheat flour and wheat bran in the above composition so that about 2 cups of roasted and ground flaxseed is mixed with about 2 cups of chick pea (garbanzo bean) flour, about 2 cups of brown rice flour, about 1 cup of raw, ground almonds, about 1 cup of raw blue agave nectar, about 1.5 cups of sunflower oil, about 0.125 teaspoons of cardamom and about 0.5 teaspoons of ginger. This provides a gluten-free nutritional supplement that nevertheless provides the nutritional benefits described above.

This ready-to-eat nutritional supplement may be ingested as a small snack or with beverages. It may also be sprinkled on or mixed with other foods, like yoghurt or hot oatmeal, or beverages, such as tea or coffee. It is suitable for individuals who wish to increase their consumption of omega-3 and omega-6 fatty acids for any of the benefits provided by these compounds, including cardiovascular health and inflammatory pain syndromes. It can also be used as a dietary supplement for weight loss especially in people who have physical disabilities which limits their daily exercise or for those with inflammation-associated diseases such as fibromyalgia.

Based upon the foregoing disclosure, it should now be apparent that the nutritional supplement will carry out the aspects set forth hereinabove. It is, therefore, to be understood that any variations evident fall within the scope of the claimed invention and thus, the selection of specific component elements can be determined without departing from the spirit of the invention herein disclosed and described.

We claim:

1. A nutritional supplement comprising:
about 2 parts roasted, ground flaxseed,
about 2 parts of chick pea flour,
about 1.5-2 parts of flour selected from the group consisting of whole wheat flour and brown rice flour,
about 1 part of raw, ground almonds, and
optionally, about 0.5-1 part of wheat bran.

2. The nutritional supplement of claim 1, additionally comprising about one part raw blue agave nectar and about 1.5-about 1.8 parts sunflower oil, wherein the proportion of liquid parts to dry parts is the same as the proportion of liquid cups to dry cups.

3. The nutritional supplement of claim 1, additionally comprising one or more of the group consisting of cardamom and ginger.

4. The nutritional supplement of claim 2, comprising about 0.0025 parts cardamom and about 0.01 parts ginger.

5. The nutritional supplement of claim 1, wherein the components are organically grown and produced.

6. The nutritional supplement of claim 5, wherein the flour is whole wheat flour and the supplement contains wheat bran.

7. The nutritional supplement of claim 5, wherein the flour is brown rice flour and the supplement does not contain whole wheat flour, wheat bran or a source of gluten.

8. A nutritional supplement comprising at least about 1.5 grams of Omega-3 fatty acids, less than about 1.5 grams (g) of glucose, and less than about 0.6 g of sucrose in a serving of about 2 ounces (about 59 milliliters (ml)).

9. The nutritional supplement of claim 8, wherein the nutritional supplement provides at least about 1.7 grams of Omega-3 fatty acids in a serving of about 2 ounces (about 59 ml).

10. The nutritional supplement of claim 6, wherein the nutritional supplement provides at least about 4 grams of Omega-6 fatty acids in a serving of about 2 ounces (about 59 ml).

11. The nutritional supplement of claim 8, wherein the nutritional supplement provides at least about 4.9 grams of Omega-6 fatty acids in a serving of about 2 ounces (about 59 ml).

12. The nutritional supplement of claim 11, wherein the nutritional supplement provides at least about 3.5 grams of dietary fiber in a serving of about 2 ounces (about 59 ml).

13. A method of supplementing the nutrition of an individual, the method comprising administering a nutritional supplement to the individual, the nutritional supplement comprising at least about 1.5 grams of Omega-3 fatty acids and containing less than about 1.5 grams (g) of glucose, and less than about 0.6 g of sucrose in a serving of about 2 ounces (about 59 ml).

14. The method of claim 13, wherein the nutritional supplement is in an unencapsulated form.

15. The method of claim 14, wherein the nutritional supplement comprises chick pea flour, ground almonds and one or more components selected from the group consisting of whole wheat flour and brown rice flour, wherein when the flour is whole wheat flour, the nutritional supplement additionally comprises wheat bran.

16. The method of claim 15, wherein the nutritional supplement additionally comprises at least one of raw blue agave nectar, and sunflower oil.

17. The method of claim 13, wherein the nutritional supplement additionally comprises at least one of raw blue agave nectar, and sunflower oil.

18. The method of claim 15, wherein the nutritional supplement comprises brown rice flour and does not contain whole wheat flour, wheat bran or a source of gluten.

19. The method of claim 15, wherein the nutritional supplement comprises whole wheat flour, and wheat bran.

20. The method of claim 19, wherein the nutritional supplement additionally comprises one or more of the group consisting of cardamom and ginger.
